(19) United States
(12) Patent Application Publication
(10) Pub. No.: US 2024/0424043 A1
(43) Pub. Date: Dec. 26, 2024

(54) COMPOSITION FOR PREVENTING OR TREATING MUSCLE DISEASE COMPRISING ARALIA ELATA EXTRACT

(71) Applicant: KOREA INSTITUTE OF SCIENCE AND TECHNOLOGY, Seoul (KR)

(72) Inventors: Myung Suk KIM, Gangneung-si (KR); Sang Hoon JUNG, Gangneung-si (KR); Hye Jin KO, Gangneung-si (KR); Tam Thi LE, Gangneung-si (KR)

(73) Assignee: KOREA INSTITUTE OF SCIENCE AND TECHNOLOGY, Seoul (KR)

(21) Appl. No.: 18/749,263

(22) Filed: Jun. 20, 2024

(30) Foreign Application Priority Data
Jun. 20, 2023 (KR) 10-2023-0079052

(51) Int. Cl. A61K 36/25; A61K 31/05; A61K 31/085; A61K 31/122; A61K 31/192; A61K 31/216; A61K 31/7048; A61P 21/00; A61P 43/00

(52) U.S. Cl. CPC A61K 36/25; A61K 31/05; A61K 31/085; A61K 31/122; A61K 31/192; A61K 31/216; A61K 31/7048; A61P 21/00; A61P 43/00

(57) ABSTRACT

The present invention relates to a composition for preventing or treating muscle disease comprising an extract of Aralia Elata. The Aralia Elata extract of the present invention has the effect of improving grip strength, increasing muscle weight, and improving exercise performance.

### TECHNICAL FIELD

This application claims benefit of priority based on Korean Patent Application No. 10-2023-0079052 filed on Jun. 20, 2023, the disclosures of which are incorporated herein by reference in their entireties.

The present invention relates to a composition for preventing or treating muscle disease comprising an extract of Aralia Elata.

### REFERENCE TO AN ELECTRONIC SEQUENCE LISTING

The contents of the electronic sequence listing (“KIST_PDPC232242US_1.xml”; Size is 21 kilobytes and it was created on Jun. 20, 2024) is herein incorporated by reference in its entirety.

### BACKGROUND ART

Muscle can be categorized into skeletal muscle, cardiac muscle, and visceral muscle, with skeletal muscle being the most abundant tissue in the human body, making up 40-45% of body weight. Skeletal muscles attach to bones via tendons and are responsible for generating bone movement or force. A muscle is made up of many myofibrils, which in turn are made up of many fibrils, which are made up of actin and myosin. When actin and myosin overlap and move, the length of the muscle shortens or lengthens, causing contraction and relaxation of the overall muscle. An increase in the size of the myofibrils means an increase in the thickness of the myofibers, resulting in muscle gain.

Muscle diseases are characterized by a progressive progression of skeletal muscle weakness that leads to impaired walking and mobility, difficulty with activities of daily living (ADLs), and loss of independence. In addition, they can lead to cardiopulmonary dysfunction and other complications, so it is important to accurately understand the characteristics of each muscle disease and approach it accordingly.

South Korea entered an aging society in 2000 with the elderly population accounting for 7.2% of the total population, and is expected to enter a super-aging society (more than 20%) in 2050 (2013 statistics on the elderly, Statistics Korea). As a person's muscle mass decreases with age (by 10-15% between the ages of 50 and 70, and by more than 30% between the ages of 70 and 80), muscle strength and muscle function also decrease, a condition known as sarcopenia. Senile sarcopenia is a major cause that limits the independent living of older people by causing activity and walking difficulties. In addition, sarcopenia lowers basal metabolic rate, increases insulin resistance, promotes type 2 diabetes, and increases the risk of high blood pressure and cardiovascular disease by 3-5 times. Currently, there are no drugs approved for the treatment of sarcopenia, and drug repositioning technology that applies myostatin inhibitors or existing FDA-approved treatments for other diseases to sarcopenia is being developed.

As a result of efforts to develop natural products effective in preventing or treating muscle diseases, the present inventors completed the present invention by experimentally confirming that Aralia Elata extract is useful for improving muscle strength and exercise performance.

### DISCLOSURE

### Technical Problem

The object of the present invention is to provide a pharmaceutical composition for preventing or treating muscle diseases comprising Aralia Elata extract as an active ingredient.

Additionally, the object of the present invention is to provide a food composition for preventing or improving/ameliorating muscle disease comprising Aralia Elata extract as an active ingredient.

Additionally, the object of the present invention is to provide a quasi-drug composition for preventing or improving/ameliorating muscle disease comprising Aralia Elata extract as an active ingredient.

However, the technical problem to be achieved by the present invention is not limited to the problems mentioned above, and other problems not mentioned will be clearly understood by those skilled in the art from the description below.

### Technical Solution

The present invention provides a food composition for preventing or ameliorating muscle disease or improving athletic performance (or athletic ability) comprising an extract of Aralia Elata (herein also refer to “Aralia Elata extract”) as an active ingredient.

In addition, the present invention provides a quasi-drug composition for preventing or ameliorating muscle disease or improving athletic performance (or athletic ability), comprising Aralia Elata extract as an active ingredient.

In addition, the present invention provides a pharmaceutical composition for preventing or treating muscle disease or improving athletic performance (or athletic ability) comprising an extract of Aralia Elata as an active ingredient.

In addition, the present invention provides a method for preventing or treating muscle disease, or a method for improving athletic performance (or athletic ability), including the step of administering or taking a composition comprising Aralia Elata extract as an active ingredient to an individual in need.

The muscle disease may be one or more diseases selected from the group consisting of sarcopenia, muscular atrophy, myasthenia, muscular dystrophy, myotonia, hypotonia, muscular weakness, muscular dystrophy, atony, amyotrophic lateral sclerosis, and inflammatory myopathy.

The composition may improve grip strength.

The composition may increase muscle weight.

The muscle may include one or more selected from the group consisting of thigh muscle, gastrocnemius muscle, calf muscle, and soleus muscle.

The composition may be extracted with a solvent selected from the group consisting of water, organic solvents, and mixtures thereof.

The organic solvent may be one or more solvents selected from the group consisting of lower alcohols having 1 to 6 carbon atoms, hexane, acetone, ethyl acetate, chloroform, and diethyl ether.

The Aralia Elata extract may contain one or more compounds selected from the group consisting of compounds represented by the following formulas 1 to 13.

### Advantageous Effects

The extract of Aralia Elata of the present invention has the effect of improving grip strength, increasing muscle weight, and improving exercise performance. In addition, the present composition comprising Aralia Elata extract as an active ingredient is a natural medicine and can be applied to various products, such as a pharmaceutical composition, food composition, or quasi-drug for preventing, treating, or improving muscle disease.

### DESCRIPTION OF DRAWINGS

FIG. 1 is a schematic diagram of the design of an animal model created to confirm the effectiveness of Aralia Elata extract in treating or ameliorating sarcopenia.

FIGS. 2A-2C show the results confirming the effects of Aralia Elata extract on increasing body weight and improving grip strength (Con: control group, Dex: Dexamethasone, Dex+S23: dexamethasone+positive control-anabolic drug 25 mg/kg, Dex+AE 100: dexamethasone+Aralia Elata extract 100 mg/kg, Dex+AE 50: Dexamethasone+Aralia Elata extract 50 mg/kg, Dex+AE 10: dexamethasone+Aralia Elata extract 10 mg/kg; **, p<0.01; ***, p<0.001).

FIGS. 3A-3C show the results confirming the effect of Aralia Elata extract on improving exercise performance.

a: maximum running speed, b: distance run until exhaustion, c: time taken until exhaustion (Con: control group, Dex: Dexamethasone, Dex+S23: dexamethasone+positive control-anabolic drug 25 mg/kg, Dex+AE 100: dexamethasone+Aralia Elata extract 100 mg/kg, Dex+AE 50: Dexamethasone+Aralia Elata extract 50 mg/kg, Dex+AE 10: dexamethasone+Aralia Elata extract 10 mg/kg; *, p<0.05).

FIGS. 4A-4D show the results confirming the effect of Aralia Elata extract on increasing muscle weight. [0029] a: thigh muscle weight, b: gastrocnemius muscle weight, c: calf muscle weight, d: soleus muscle weight (Con: control group, Dex: Dexamethasone, Dex+S23: dexamethasone+positive control-anabolic drug 25 mg/kg, Dex+AE 100: dexamethasone+Aralia Elata extract 100 mg/kg, Dex+AE 50: Dexamethasone+Aralia Elata extract 50 mg/kg, Dex+AE 10: dexamethasone+Aralia Elata extract 10 mg/kg; ** p<0.01; p<0.001).

FIG. 5 shows the results of confirming the effectiveness of 13 compounds isolated from Aralia Elata extract in ameliorating the survival rate of muscle cells reduced by dexamethasone (Con: Control, Dex: Dexamethasone, 1: 1-ethyl-3,4,6-trihydroxy-5H-benzocyclohepten-5-one, 2: 3,4,6-trihydroxy-5H-benzocyclohepten-5-one, 3: Catechol, 4: 4-ethyl catechol, 5: Protocatechuic acid, 6: Caffeic acid, 7: Dihydroxycaffeic acid, 8: Phloretic acid, 9: Methyl dihydroxycaffeate, 10: Junipediol A, 11: Chlorogenic acid, 12: Neochlorogenic acid, 13: Quercetin 7-O-neohesperidoside; *, p<0.05; **p<0.01; ***, p<0.005).

FIG. 6 shows the results of confirming the average myofiber enhancement effect of three compounds isolated from Aralia Elata extract (Con: Control, Dex: Dexamethasone, Dex+Catechol: Dexamethasone+Catechol, Dex+4-EC: Dexamethasone+4-ethyl catechol, Dex+MD: Dexamethasone+Methyl Dihydroxycaffeate; *, p<0.05; **p<0.01; ***, p<0.005).

FIG. 7 shows the results confirming the effectiveness of three compounds isolated from Aralia Elata extract to enhance muscle differentiation-related gene expression (Con: Control, Dex: Dexamethasone, Dex+Catechol: Dexamethasone+Catechol, Dex+4-EC: Dexamethasone+4-ethyl catechol, Dex+MD: Dexamethasone+Methyl Dihydroxycaffeate; p<0.05; **p<0.01; ***, p<0.005).

FIG. 8 shows the results confirming the efficacy of three compounds isolated from Aralia Elata extract to improve oxidative stress and mitochondrial function (Con: Control, Dex: Dexamethasone, Dex+Catechol: Dexamethasone+Catechol, Dex+4-EC: Dexamethasone+4-ethyl catechol, Dex+MD: Dexamethasone+Methyl Dihydroxycaffeate; *, p<0.05; **p<0.01; ***, p<0.005).

FIG. 9 shows the results confirming the effectiveness of three compounds isolated from Aralia Elata extract to enhance gene expression related to exercise performance (Con: Control, Dex: Dexamethasone, Dex+Catechol: Dexamethasone+Catechol, Dex+4-EC: Dexamethasone+4-ethyl catechol, Dex+MD: Dexamethasone+Methyl Dihydroxycaffeate; p<0.05; **p<0.01; ***, p<0.005).

### MODE OF THE INVENTION

The present inventors completed the present invention by experimentally confirming that the extract of Aralia Elata, a natural product with little or no side effects, is effective in increasing muscle weight and improving grip strength and exercise performance.

Hereinafter, the present invention will be described in detail.

The present invention provides a composition for preventing, ameliorating or treating muscle disease comprising Aralia Elata extract as an active ingredient.

The above muscle disease can be one or more diseases selected from the group consisting of sarcopenia, muscular atrophy, myasthenia, muscular dystrophy, myotonia, hypotonia, muscular weakness, muscular dystrophy, atony, amyotrophic lateral sclerosis, and inflammatory myopathy.

The composition can improve grip strength.

The composition can increase exercise performance.

The composition can increase muscle weight.

The muscle can comprise one or more selected from the group consisting of thigh muscle, gastrocnemius muscle, calf muscle, and soleus muscle.

The Aralia Elata extract may contain one or more compounds selected from the group consisting of compounds represented by the following formulas 1 to 13.

More preferably, the Aralia Elata extract may include one or more compounds selected from the group consisting of compounds represented by Formula 3, Formula 4, and Formula 9 below:

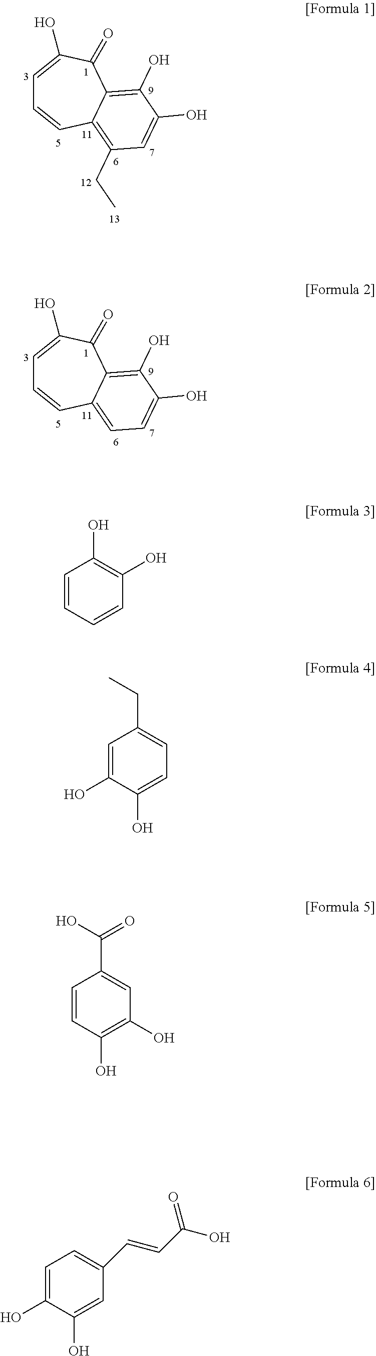

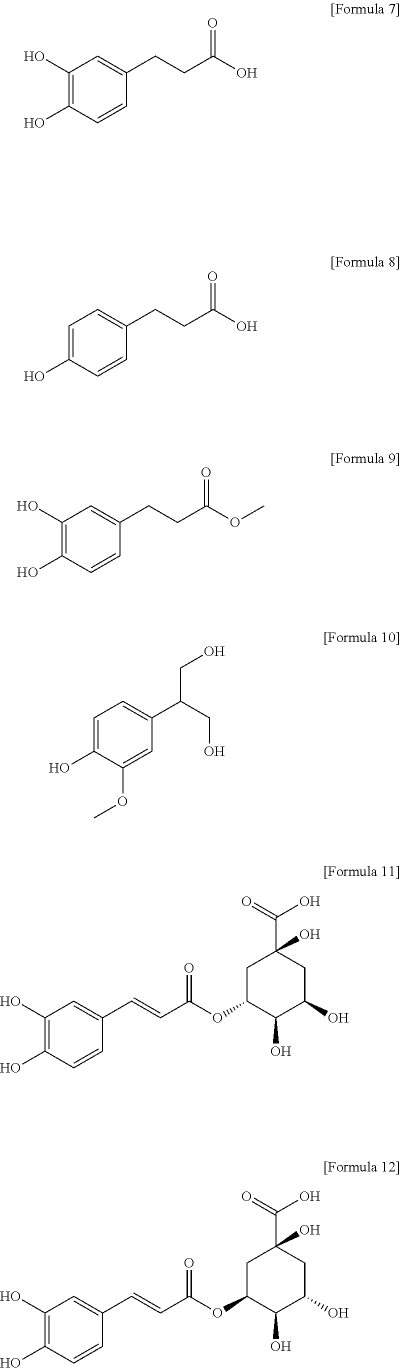 [SMILES: O=C(O)CCc1ccc(O)c(O)c1 ; O=C(O)CCc1ccc(O)cc1 ; COC(=O)CCc1ccc(O)c(O)c1 ; COc1cc(C(CO)CO)ccc1O ; O=C(/C=C/c1ccc(O)c(O)c1)O[C@@H]1C[C@](O)(C(=O)O)C[C@@H](O)[C@H]1O ; O=C(/C=C/c1ccc(O)c(O)c1)O[C@H]1C[C@](O)(C(=O)O)C[C@H](O)[C@H]1O]

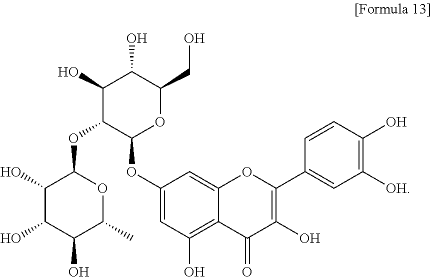 [SMILES: C[C@H]1O[C@H](O[C@H]2[C@H](Oc3cc(O)c4c(=O)c(O)c(-c5ccc(O)c(O)c5)oc4c3)O[C@H](CO)[C@@H](O)[C@@H]2O)[C@@H](O)[C@@H](O)[C@@H]1O]

The composition can be a food composition, a health functional food composition, a pharmaceutical composition, or a quasi-drug composition.

As used herein, “preventing” means any act of delaying the onset of a muscle disease by administration of a composition of the invention, and “treating” and “ameliorating/improving” means any act of ameliorating or beneficially altering the symptoms of a muscle disease by administration of a composition of the invention.

The Aralia Elata extract can be extracted using a polar solvent or/and a non-polar solvent.

The polar solvent may comprise one or more selected from the group consisting of (i) water, (ii) alcohol (preferably, methanol, ethanol, propanol, butanol, normal-propanol, iso-propanol, normal-butanol, 1-pentanol, 2-butoxyethanol or ethylene glycol), (iii) acetic acid, (iv) dimethyl-formamide (DMFO), and (v) dimethyl sulfoxide (DMSO). The non-polar solvent may include at least one selected from the group consisting of acetone, acetonitrile, ethyl acetate, methyl acetate, fluoroalkanes, pentane, hexane, 2,2,4-trimethylpentane, decane, cyclohexane, cyclopentane, diisobutylene, 1-pentene, 1-chlorobutane, 1-chloropentane, o-xylene, diisopropyl ether, 2-chloropropane, toluene, 1-chloropropane, chlorobenzene, benzene, diethyl ether, diethyl sulfide, chloroform, dichloromethane, 1,2-dichloroethane, aniline, diethylamine, ether, carbon tetrachloride, and THF.

More specifically, the extract may be extracted using water, an organic solvent, or a mixture thereof as a solvent, and according to certain embodiments of the present invention, the extract of the present invention may be obtained by treating Aralia Elata with water.

The organic solvent may comprise one or more solvents selected from the group consisting of lower alcohols (methanol, ethanol, propanol, butanol, etc.) having a carbon number of 1 to 6, hexane, acetone, ethyl acetate, chloroform, and diethyl ether.

Furthermore, the extract may be prepared as a fraction according to methods conventionally used in the art, and the solvent used to fractionate the extract may be one or more selected from the group consisting of, but not limited to, water, lower alcohols of C1 to C4, n-hexane, ethyl acetate, acetone, acetonitrile, butyl acetate, 1,3-butylene glycol, methylene chloride, and mixtures thereof. However, a butanol fraction may be more preferred.

As used herein, the term “extract” has the meaning commonly understood in the art as a crude extract as described above, but broadly includes fractions obtained by further fractionation of the extract, i.e., the extract is not only obtained using the extraction solvent described above, but also includes fractions obtained by further purification. For example, fractions obtained by passing the extract through an ultrafiltration membrane with a certain molecular weight cut-off value, separation by various chromatographies (designed for separation based on size, charge, hydrophobicity or affinity), separation by different chromatographic techniques, etc.

The extract used in the present invention can be prepared in powder form by additional processes such as reduced pressure distillation and freeze drying or spray drying.

Specifically, the present extract can be extracted by adding water, an organic solvent, or a mixture thereof, to Aralia Elata or leaves of Aralia Elata in an amount of 5 times to 50 times the weight of Aralia Elata or leaves of Aralia Elata, more preferably in an amount of 10 times to 30 times the weight of Aralia Elata or leaves of Aralia Elata, but not limited to. The extraction temperature may be 10° C. to 150° C., and is more preferably 15° C. to 120° C., but is not limited thereto. The extraction time may be preferably from 1 hour to 20 hours, and more preferably from 2 hours to 8 hours, but is not limited thereto. The extraction method may be, but is not limited to, cold immersion, ultrasonic extraction, or reflux cooling extraction. The number of extraction times may be preferably from 1 to 5, and more preferably from 2 to 3 repeated extractions. Further, the extract may be diluted, concentrated, or diluted or concentrated and then purified and dried for use.

As used herein, the term ‘comprising as an active ingredient’ means containing a sufficient amount to achieve the efficacy or activity of the Aralia Elata extract. The present invention is a composition comprising an extract(s) from Aralia Elata, a natural plant material, and the upper quantitative limit of the Aralia Elata extract contained in the composition of the present invention can be selected and implemented by a person skilled in the art within an appropriate range.

### Pharmaceutical Composition for Preventing or Treating Muscle Disease, or Improving Athletic Performance Comprising Aralia Elata Extract as an Active Ingredient

The composition of the present invention can be prepared as a pharmaceutical composition.

When the composition of the present invention is formulated as a pharmaceutical composition, the pharmaceutical composition of the present invention may comprise a pharmaceutically acceptable carrier.

According to a preferred embodiment of the present invention, the composition of the present invention contains (a) a pharmaceutically effective amount of the above-described Aralia Elata extract of the present invention; and (b) a pharmaceutically acceptable carrier. As used herein, the term “pharmaceutically effective amount” refers to an amount sufficient to achieve the efficacy or activity of the above-described Aralia Elata extract.

Pharmaceutically acceptable carriers are those customarily used in the formulation: lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oils, but not limited thereto. In addition to the above ingredients, the pharmaceutical composition of the present invention may further include lubricants, wetting agents, sweeteners, flavoring agents, emulsifiers, suspending agents, preservatives, etc.

Suitable pharmaceutically acceptable carriers and formulations are described in detail in Remington's Pharmaceutical Sciences (19th ed., 1995).

The pharmaceutical composition of the present invention can be administered orally or parenterally.

Suitable dosages of the pharmaceutical composition of the present invention can be prescribed in a variety of ways, depending on factors such as the method of formulation, mode of administration, patient age, weight, sex, medical condition, food, time of administration, route of administration, rate of excretion, and response sensitivity. Typical dosages of the active ingredients in the pharmaceutical composition of the present invention are in the range of 0.001-100 mg/kg/day, preferably 0.01-35 mg/kg/day in adults. The dose may be administered once daily or may be divided into several doses. However, the above dosages do not limit the scope of the present invention.

The pharmaceutical composition of the present invention may be prepared in unit dose form or by formulation with pharmaceutically acceptable carriers and/or excipients, in accordance with methods readily practiced by a person of ordinary skill in the art to which the invention belongs. The formulation may be in the form of a solution, suspension, syrup, or emulsion in an oil or aqueous medium, or in the form of an excipient, acid, powder, granule, tablet, or capsule, and may further comprise a dispersant or stabilizing agent.

### Quasi-Drug Composition for Preventing or Ameliorating Muscle Disease, or Improving Athletic Performance Comprising Aralia Elata Extract as an Active Ingredient Composition

The composition of the present invention may be provided as a quasi-drug composition.

The Aralia Elata extract may be added as is, or may be used together with ingredients such as other quasi-drugs, and may be used appropriately according to conventional methods.

The mixing amount of the active ingredient can be appropriately determined depending on the purpose of use (prevention, health, or therapeutic treatment). The quasi-drug composition may be used in the manufacture of external preparations, patches, ointments, etc., but is not limited thereto.

### Food Composition for Preventing or Ameliorating Muscle Disease, or Improving Athletic Performance Comprising Aralia Elata Extract as an Active Ingredient

The composition of the present invention may be provided as a food composition or health functional food composition.

When the composition for preventing, improving/ameliorating, or treating muscle disease, or improving athletic performance containing the Aralia Elata extract of the present invention is manufactured as a food composition, it may include not only the extract of the present invention, but also ingredients that are customarily added in the manufacture of food products, for example, proteins, carbohydrates, fats, nutrients, seasonings and flavorings. Examples of the carbohydrates described above are monosaccharides, e.g., glucose, fructose, and the like; disaccharides, e.g., maltose, sucrose, oligosaccharides, and the like; and polysaccharides, e.g., dextrins, cyclodextrins, and the like, and sugar alcohols, e.g., xylitol, sorbitol, erythritol, and the like. As flavoring agents, natural flavoring agents [such as thaumatin, stevia extract (e.g., rebaudioside A, glycyrrhizin, etc.)] and synthetic flavoring agents (such as saccharin, aspartame, etc.) can be used. For example, if the food composition of the present invention is formulated as a beverage, it may additionally contain citric acid, liquid dextrose, sugar, glucose, acetic acid, malic acid, fruit juice, caterpillar extract, jujube extract, licorice extract, etc. in addition to the natural product extract of the present invention.

The formulation of the food composition or dietary supplement composition may be in the form of an acid, granule, pill, tablet, capsule, or capsule, as well as in the form of a conventional food or beverage.

Examples of food products to which the substance may be added include, without limitation, meat, sausages, bread, chocolate, candy, snacks, confectionery, pizza, ramen, other noodles, chewing gum, dairy products including ice cream, various soups, beverages, teas, drinks, alcoholic beverages and vitamin complexes, all of which are food products in the ordinary sense of the term.

In general, when manufacturing food or beverages, the Aralia Elata extract can be added in an amount of 15 parts by weight or less, preferably 10 parts by weight or less, based on 100 parts by weight of raw materials. However, in the case of long-term intake for the purpose of health and hygiene or health control, the amount may be below the above range, and the present invention may also be used in amounts above the above range, as the invention is safe in that it utilizes natural products

Hereinafter, the present invention will be described in more detail through examples. The purpose, features, and advantages of the present invention will be easily understood through the following examples. The present invention is not limited to the embodiments described herein and may be embodied in other forms. The embodiments introduced here are provided to enable the idea of the present invention to be sufficiently conveyed to those skilled in the art. Therefore, the present invention should not be limited by the following examples.

### [Preparation Example 1] Preparation of Aralia Elata Extract

The leaves of Aralia Elata were purchased from Herb Village Co., Ltd. (Korea), and cut into appropriate sizes. 2 kg of the cut leaves of Aralia Elata and 10 L of water were added to the extraction vessel for cold-extraction for 3 days, and the sediment was removed using a centrifuge, and the impurities were filtered to obtain the filtrate. Afterwards, the solvent was concentrated and dried under reduced pressure to obtain 310 g of the present extract of Aralia Elata.

### [Preparation Example 2] Isolation of Compounds Derived from Aralia Elata Extract

The extract obtained according to Preparation Example 1 was subjected to solvent fractionation and the compound was separated. Specifically, the extract of Preparation Example 1 was suspended with distilled water (10 L×3 times) and then an equal amount of butanol was added to separate it into a butanol layer and a water layer, which was filtered and concentrated under reduced pressure to obtain a butanol fraction (40 g) and a water fraction (260 g). The butanol fraction was then separated by MC: MeOH=15:1-0:15 using a silica gel open column and separated by molecular weight using a Sephadex open column under 50% methanol, and 13 compounds in Table 1 were obtained by prep-MPLC.

TABLE 1
Compounds derived from Aralia Elata extract

| No. | Compound |
|---|---|
| 1 (Formula 1) | 1-Ethyl-3,4,6-trihydroxy-5H-benzocyclohepten-5-one |
| 2 (Formula 2) | 3,4,6-trihydroxy-5H-benzocyclohepten-5-one |
| 3 (Formula 3) | Catechol |
| 4 (Formula 4) | 4-ethyl catechol |
| 5 (Formula 5) | Protocatechuic acid |
| 6 (Formula 6) | Caffeic acid |
| 7 (Formula 7) | Dihydroxycaffeic acid |
| 8 (Formula 8) | Phloretic acid |
| 9 (Formula 9) | Methyl dihydroxycaffeate |
| 10 (Formula 10) | Junipediol A |
| 11 (Formula 11) | Chlorogenic acid |
| 12 (Formula 12) | Neochlorogenic acid |
| 13 (Formula 13) | Quercetin 7-O-neohesperidoside |

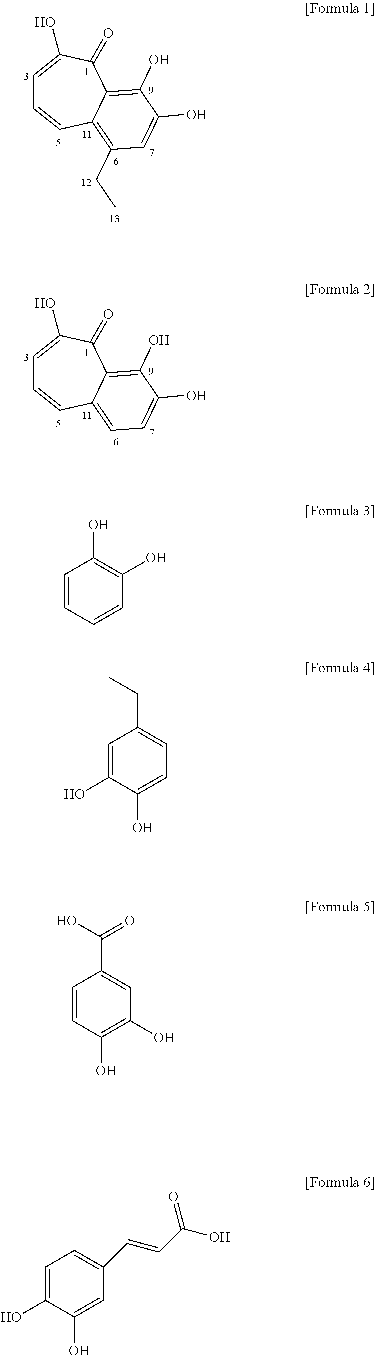

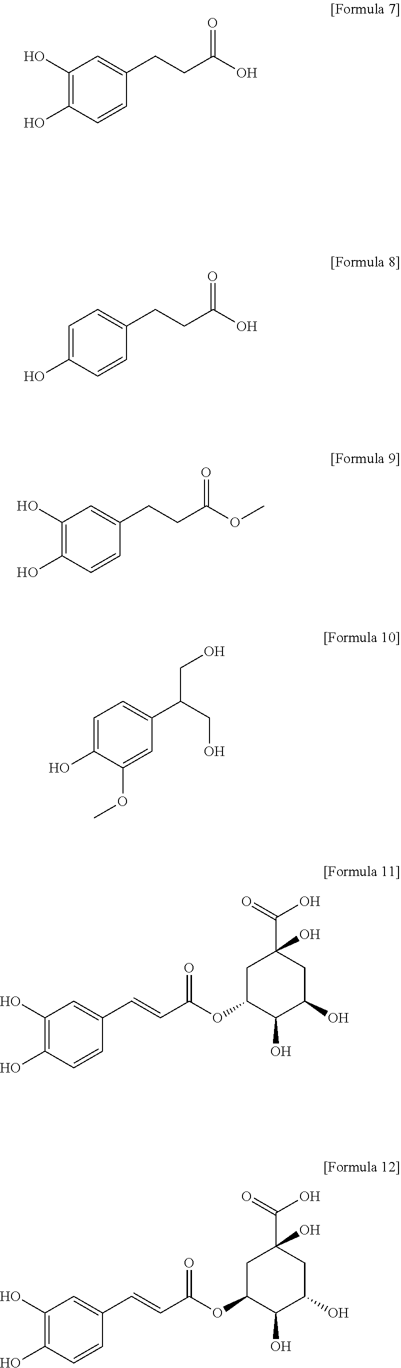 [SMILES: O=C(O)CCc1ccc(O)c(O)c1 ; O=C(O)CCc1ccc(O)cc1 ; COC(=O)CCc1ccc(O)c(O)c1 ; COc1cc(C(CO)CO)ccc1O ; O=C(/C=C/c1ccc(O)c(O)c1)O[C@@H]1C[C@](O)(C(=O)O)C[C@@H](O)[C@H]1O ; O=C(/C=C/c1ccc(O)c(O)c1)O[C@H]1C[C@](O)(C(=O)O)C[C@H](O)[C@H]1O]

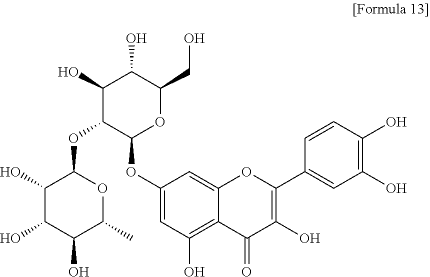 [SMILES: C[C@H]1O[C@H](O[C@H]2[C@H](Oc3cc(O)c4c(=O)c(O)c(-c5ccc(O)c(O)c5)oc4c3)O[C@H](CO)[C@@H](O)[C@@H]2O)[C@@H](O)[C@@H](O)[C@@H]1O]

### [Example 1] Confirmation of Changes in Grip Strength and Body Weight of Mice in which Sarcopenia was Induced by Administration of Dexamethasone Using Aralia Elata Extract

The present inventors created a sarcopenia mouse model to confirm the effectiveness of Aralia Elata extract prepared in the above-described preparation example to treat or improve sarcopenia, and measured grip strength using the control mouse and the sarcopenia mouse model.

The mice used to confirm the efficacy of Aralia Elata extract were prepared through the following process.

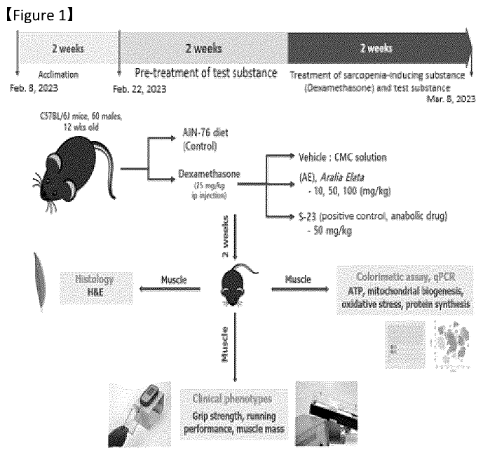

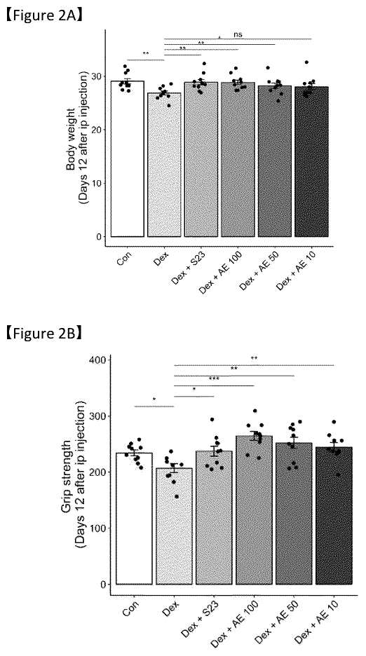

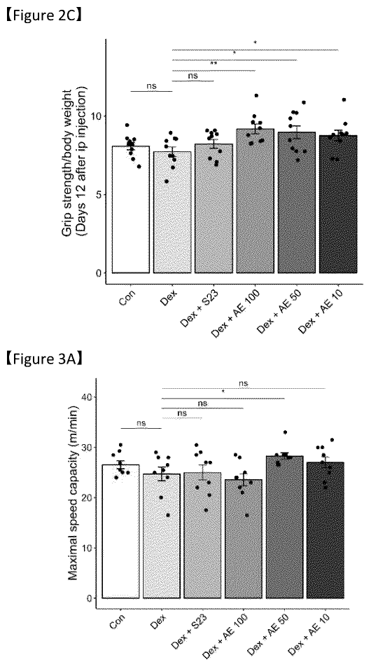

As shown in FIG. 1, C57BL/6J mice (male, 12 weeks old) were obtained from Duyeol Biotech Co., Ltd., and after an adaptation period of 2 weeks, they were divided into 6 test groups (n=10 per group) according to body weight. The diet, dose, and method of administration administered to the experimental group are shown in Table 2 below. The test substance was prepared in the form of a liquid suspension using 0.5% CMC and administered for 4 weeks. To correct for the placebo effect of 0.5% CMC and the weight loss effect due to administration stress, 0.5% CMC was orally administered daily to the vehicle control group. Two weeks after administration of the test substance, the experimental group was administered intraperitoneal injection of Dexamethasone for two weeks to induce sarcopenia, and the normal control group was administered saline solution by intraperitoneal injection. All mice were provided with the same AlN76 diet (Research Diets), and the test substances were administered for an additional 2 weeks for a total of 4 weeks. The dark: light cycle was maintained at 12-hour:12-hour intervals, and water was allowed to be consumed freely. As a result of the experiment, as shown in FIG. 2, it was confirmed that grip strength and body weight decreased in the Dexamethasone (Dex) group over time, and in the positive control group of mice fed anabolic drug and Aralia Elata extract, grip strength and body weight increased compared to the group treated only with Dexamethasone. (Dex: Dexamethasone sarcopenia-inducing substance (25 mg/kg), AE: Aralia Elata extract (10, 50, or 100 mg/kg)).

TABLE 2

| | | Sarcopenia- | Orally | Dosage and | |
|---|---|---|---|---|---|
| | | inducing | administered | method of | Administration |
| Test group | Feed | substance | substance | administration | period |
| 1(Con) | AIN76 | Saline | Vehicle | — | After 2 weeks |
| | diet | | | | of oral |
| | | | | | administration |
| 2(Dex) | | Dexamethasone | Vehicle | — | of test |
| | | (25 mg/kg, once | | | substance, |
| 3 (Dex + | | a day, 2 weeks) | anabolic drug | 25 mg/kg, | 2 weeks of |
| S23) | | | (S-23) | once daily, | administration |
| 4 (Dex + | | | Aralia Elata | 100 mg/kg, | of test |
| AE 100) | | | extract | once daily | substance and |
| 5 (Dex + | | | | 50 mg/kg, | inducing |
| AE 50) | | | | once daily | substance |
| 6 (Dex + | | | | 10 mg/kg, | (Total 4 |
| AE 10) | | | | once daily | weeks) |

### [Example 2] Confirmation of the Efficacy of Improving Exercise Performance in Mice in which Sarcopenia was Induced by Dexamethasone Administration Using Aralia Elata Extract

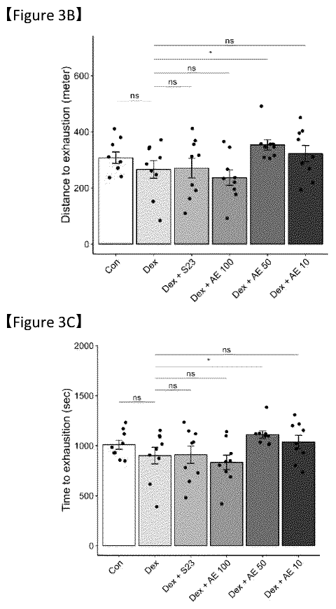

In [Example 1], after administration of the Aralia Elata extract for a total of 4 weeks, exercise performance was evaluated for all administration groups. Specifically, maximum running speed, distance run until exhaustion, and period until exhaustion were evaluated, and the results are shown in FIG. 3. As shown in FIG. 3, it was confirmed that exercise performance significantly increased in the group administered Aralia Elata extract.

### [Example 3] Confirmation of Weight Increase by Muscle Type in Mice in which Sarcopenia was Induced by Administration of Dexamethasone Using Aralia Elata Extract

In [Example 1], after administration of the Aralia Elata extract for a total of 4 weeks, the weight of each type of muscle tissue in all administration groups was confirmed.

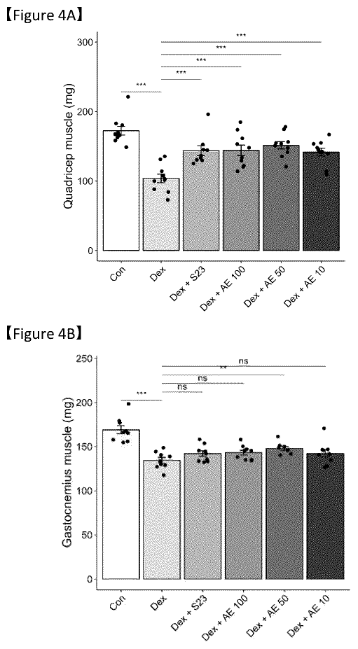

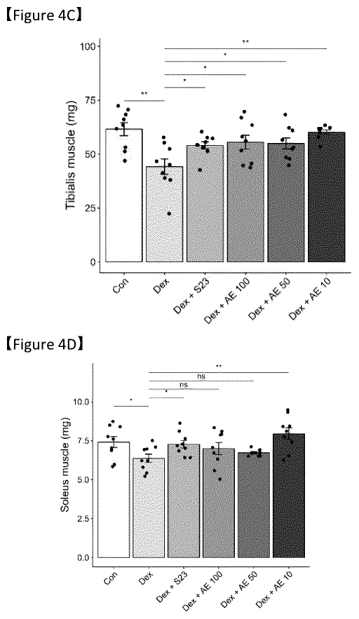

As a result, as shown in FIG. 4, it was confirmed that the weight of the thigh muscle, gastrocnemius muscle, calf muscle, and soleus muscle were decreased in the Dexamethasone-administered group compared to the saline-administered control group, but when Aralia Elata extract was administered, the weight of these muscles were significantly increased.

### [Example 4] Confirmation of the Efficacy of Compounds Derived from Aralia Elata Extract to Improve Myotube Cell Survival Rate

Cell culture and differentiation of myoblasts: C2C12 mouse (CRL1772; American type cell collection, USA) derived myoblasts were cultured in Dulbecco's Modified Eagle's Medium (DMEM) medium supplemented with 10 (v/v) % FBS, and after reaching 90% confluence, they were transferred to DMEM medium containing 2 (v/v) % horse serum and cultured for 7 days to differentiate into myotubes.

C2C12 myoblasts were differentiated into myotubes, and then treated with 100 μM of dexamethasone for 24 hours to induce myoatrophy, and 20 μM of 13 compounds isolated from the extract (see Table 1) to determine the effect of alleviating myoatrophy. As shown in FIG. 5, the results confirmed that the treatment with the compounds isolated from the extract restored the viability of myotubes that was reduced by dexamethasone treatment. In particular, the compound 3 (catechol), compound 4 (4-ethyl catechol), and compound 9 (methyl dihydroxycaproate) treatment groups showed a significantly superior effect.

### [Example 5] Confirmation of the Efficacy of Compounds Derived from Aralia Elata Extract in Enhancing the Thickness of Myotubes

As in Example 4, C2C12 myoblasts were differentiated into myotubes and then treated with 100 μM of dexamethasone for 24 hours to induce myoatrophy and 20 μM of three compounds (catechol, 4-ethyl catechol, and methyl dihydroxycaffeate) isolated from the extract of Aralia Elata to determine changes in myotube thickness.

Changes in thickness of myotubes were determined as follows. Cells were washed twice with phosphate buffered saline (PBS) and fixed with 4% formaldehyde for 15 minutes. After permeabilization with 0.4% Triton-X100 in DPBS for 10 minutes and blocking with 10% donkey serum prepared in 0.1% Triton X-100, 1% BSA/DPBS for 1 hour, cells were stained with 1:100 total MHC (MF20, Developmental Research Hybridoma Bank) overnight at 4° C. After washing three times with 0.1% Triton-X100/DPBS, cells were incubated with FITC fluorochrome secondary antibody (1:100) at Hoechst 1 uM prepared in 0.1% Triton X-100, 1% BSA/DPBS for 1 hour at room temperature. After washing three times with 0.1% Triton-X100/DPBS, the immunofluorescence of C2C12 myotube was observed and myofibers were quantified using an Operetta machine 20 at 10× magnification (3 replicates/group).

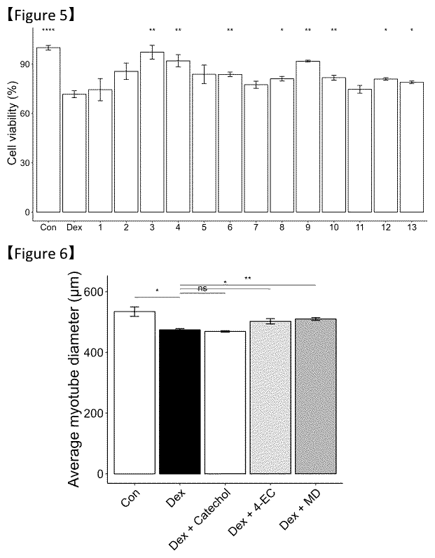

As shown in FIG. 6, an increase in the thickness of myotubes, which was reduced by the dexamethasone treatment, was observed in the compounds isolated from the extract of Aralia Elata extract.

### [Example 6] Confirmation of the Efficacy of Compounds Derived from Aralia Elata Extract to Enhance Gene Expression Related to Muscle Cell Differentiation

C2C12 mouse-derived myotubes treated with dexamethasone 10 μM were washed twice with PBS, and total RNA was extracted according to the user-recommended protocol of an RNA extraction kit (GeneAll RNA isolation kit, Seoul, South Korea). cDNA was synthesized from the extracted RNA and subjected to real time-quantitative polymerase chain reaction (RT-qPCR). POR analysis was performed according to the user-recommended cycling conditions of the analyzer (Applied Biosystems 7900 HT thermal cycler, Applied Biosystems). To determine the expression level of each mRNA, fold change values relative to the control were calculated using beta-actin as an endogenous control, and relative expression levels were compared. The primer sequences are as follows:

TABLE 3

| | Primer | | Sequence |
|---|---|---|---|
| Gene | type | Sequence | number |
| Mstn | forward | TGG CTC CTA CTG GAC CTC TC | SEQ ID |
| | | | NO: 1 |
| | reverse | AAG ATG CAG TCA CTC CC | SEQ ID |
| | | | NO: 2 |
| Myd1 | forward | CAT AGA CTT GAC AGG CCC CG | SEQ ID |
| | | | NO: 3 |
| | reverse | CGG GTC CAG GTC CTC AAA AA | SEQ ID |
| | | | NO: 4 |
| Myog | forward | AGC TAT CCG GTT CCA AAG CC | SEQ ID |
| | | | NO: 5 |
| | reverse | GCA CAG GAG ACC TTG GTC AG | SEQ ID |
| | | | NO: 6 |
| Myf5 | forward | TCC AGG TAT TCC CAC CTG CT | SEQ ID |
| | | | NO: 7 |
| | reverse | TCA GCT TTG TGT GCT CCG AA | SEQ ID |
| | | | NO: 8 |

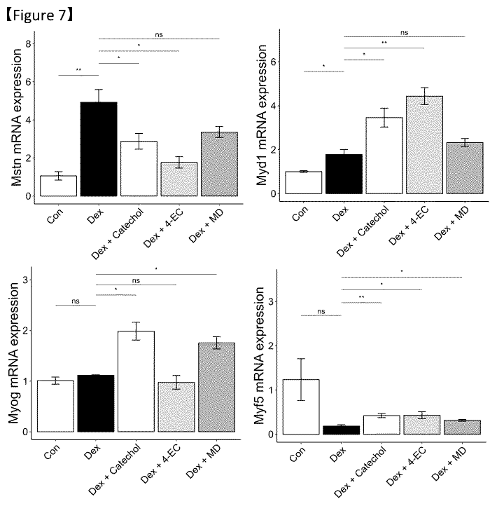

As a result, Mstn gene expression, a marker of muscle growth inhibition, which was increased by dexamethasone treatment, was decreased by treatment with the present compounds (catechol, 4-ethyl catechol, and methyl dihydroxycaffeate) derived from Aralia Elata extract. In addition, it was confirmed that that Myd1, Myog, and Myf5 gene expression, markers of myogenic differentiation, which were reduced by dexamethasone treatment, were increased by treatment with the present compounds (catechol, 4-ethyl catechol, and methyl dihydroxycaffeate) derived from the extract of Aralia Elata (FIG. 7).

### [Example 7] Confirmation of the Efficacy of Compounds Derived from Aralia Elata Extract to Improve Oxidative Stress and Mitochondrial Function

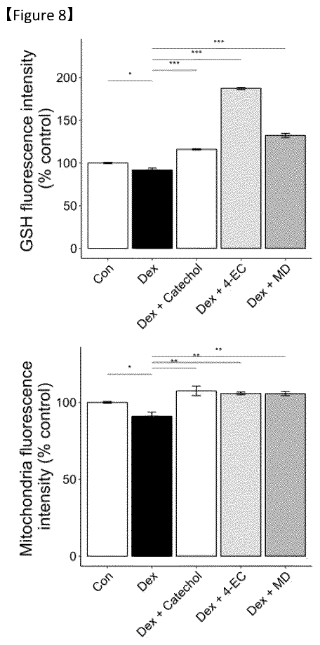

As in Example 4, C2C12 myoblasts were differentiated into myotubes, and then treated with 100 μM of dexamethasone for 24 hours to induce oxidative stress or mitochondrial dysfunction, and then treated with 20 μM of three compounds (catechol, 4-ethyl catechol, and methyl dihydroxycaffeate) isolated from the extract of Aralia Elata to determine their efficacy in ameliorating oxidative stress and mitochondrial dysfunction. The results showed that the glutathione (GSH) content, which has antioxidant activity, was decreased by dexamethasone treatment, but the GSH content was increased again by treatment with the present compounds derived from the extract of Aralia Elata. Furthermore, the mitochondrial dysfunction induced by dexamethasone treatment was ameliorated by treatment with the present compound derived from the extract of Aralia Elata (FIG. 8).

### [Example 8] Confirmation of the Efficacy of Compounds Derived from Aralia Elata Extract to Enhance Gene Expression Related to Exercise Performance

C2C12 mouse-derived endodermal cells treated with dexamethasone 10 μM were washed twice with PBS, and total RNA was extracted according to the user-recommended protocol of an RNA extraction kit (GeneAll RNA isolation kit, Seoul, South Korea). cDNA was synthesized from the extracted RNA, and subjected to real time-quantitative polymerase chain reaction (RT-qPCR). PCR analysis was performed according to the user-recommended cycling conditions of the analyzer (Applied Biosystems 7900 HT thermal cycler, Applied Biosystems). To determine the expression level of each mRNA, fold change values relative to the control were calculated using beta-actin as an endogenous control, and relative expression levels were compared. The primer sequences are as follows:

TABLE 4

| | Primer | | Sequence |
|---|---|---|---|
| Gene | type | Sequence | number |
| Ppargc1a | forward | GTT GCC TGC ATG AGT | SEQ ID |
| | | GTG TG | NO: 9 |
| | reverse | CAC ATG TCC CAA GCC | SEQ ID |
| | | ATC CA | NO: 10 |
| Ucp3 | forward | GTT TTG CGG ACC TCC | SEQ ID |
| | | TCA CT | NO: 11 |
| | reverse | CTC TGT GCG CAC CAT | SEQ ID |
| | | AGT CA | NO: 12 |
| Tomm20 | forward | TGT GCG GTG TGT CTG | SEQ ID |
| | | TT | NO: 13 |
| | reverse | TAA GTG CCC AGA GCA | SEQ ID |
| | | CAG GA | NO: 14 |
| Tfam | forward | GGG AAT GTG GAG CGT | SEQ ID |
| | | GCT AA | NO: 15 |
| | reverse | TGA TAG ACG AGG GGA | SEQ ID |
| | | TGC GA | NO: 16 |

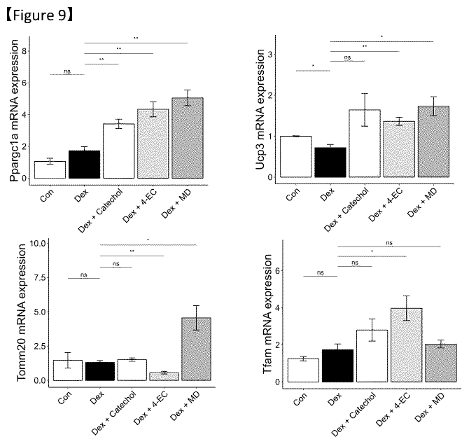

It was confirmed that Ppargc1a, UCp3, Tomm20, and Tfam gene expressions, which were reduced by dexamethasone treatment, were increased by treatment with the present compounds derived from Aralia Elata extract (catechol, 4-ethyl catechol, and methyl dihydroxycaffeate) (FIG. 9).

1. A food composition for preventing or ameliorating muscle disease or improving athletic performance comprising Aralia Elata extract as an active ingredient.

2. The food composition of claim 1, wherein the muscle disease is one or more diseases selected from the group consisting of sarcopenia, muscular atrophy, myasthenia, muscular dystrophy, sarcopenia, hypotonia, and muscular weakness, muscular dystrophy, atony, myoatrophic lateral sclerosis, and inflammatory myopathy.

3. The food composition of claim 1, wherein the composition increases a grip strength.

4. The food composition of claim 1, wherein the composition increases muscle weight.

5. The food composition of claim 4, wherein the muscle is one or more muscle selected from the group consisting of thigh muscle, gastrocnemius muscle, calf muscle, and soleus muscle.

6. The food composition of claim 1, wherein the composition is extracted with a solvent selected from the group consisting of water, organic solvents, and mixtures thereof.

7. The food composition of claim 6, wherein the organic solvent is one or more solvents selected from the group consisting of lower alcohols having 1 to 6 carbon atoms, hexane, acetone, ethyl acetate, chloroform, and diethyl ether.

8. The food composition of claim 1, wherein the Aralia Elata extract comprises at least one selected from the group consisting of compounds represented by the following formulas 1 to 13:

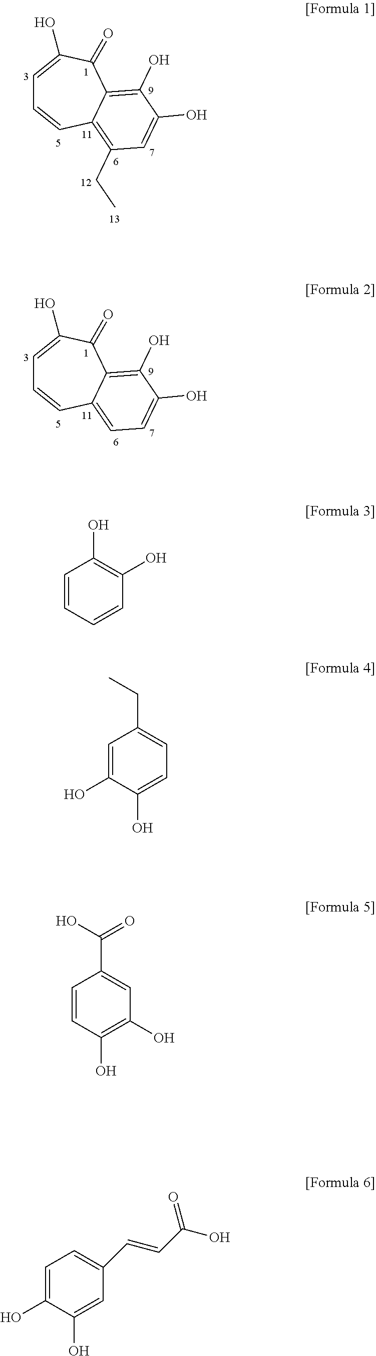

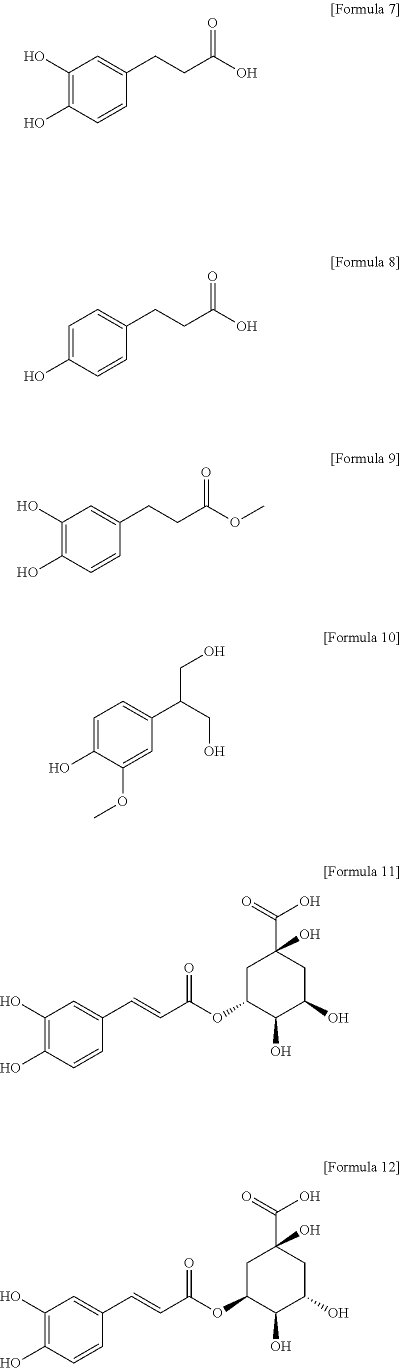 [SMILES: O=C(O)CCc1ccc(O)c(O)c1 ; O=C(O)CCc1ccc(O)cc1 ; COC(=O)CCc1ccc(O)c(O)c1 ; COc1cc(C(CO)CO)ccc1O ; O=C(/C=C/c1ccc(O)c(O)c1)O[C@@H]1C[C@](O)(C(=O)O)C[C@@H](O)[C@H]1O ; O=C(/C=C/c1ccc(O)c(O)c1)O[C@H]1C[C@](O)(C(=O)O)C[C@H](O)[C@H]1O]

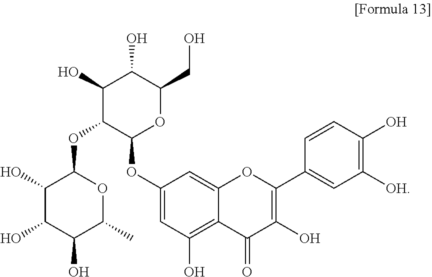 [SMILES: C[C@H]1O[C@H](O[C@H]2[C@H](Oc3cc(O)c4c(=O)c(O)c(-c5ccc(O)c(O)c5)oc4c3)O[C@H](CO)[C@@H](O)[C@@H]2O)[C@@H](O)[C@@H](O)[C@@H]1O]

9. A quasi-drug composition for preventing or ameliorating muscle disease or improving athletic performance comprising Aralia Elata extract as an active ingredient composition.

10. A method for preventing or treating muscle disease, or a method for improving athletic performance, comprising administering or taking a composition comprising Aralia Elata extract as an active ingredient to an individual in need.

11. The method of claim 10, wherein the muscle disease is one or more diseases selected from the group consisting of sarcopenia, muscular atrophy, myasthenia, muscular dystrophy, sarcopenia, hypotonia, and muscular weakness, muscular dystrophy, atony, myoatrophic lateral sclerosis, and inflammatory myopathy.

12. The method of claim 10, wherein the composition increases a grip strength.

13. The method of claim 10, wherein the composition increases muscle weight.

14. The method of claim 13, wherein the muscle is one or more muscle selected from the group consisting of thigh muscle, gastrocnemius muscle, calf muscle, and soleus muscle.

15. The method of claim 10, wherein the Aralia Elata extract comprises at least one selected from the group consisting of compounds represented by the following formulas 1 to 13: 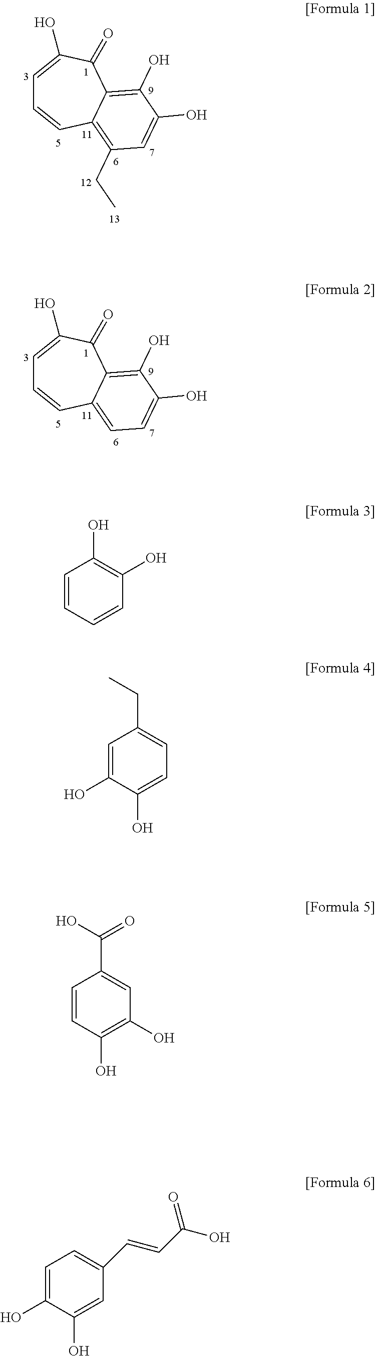 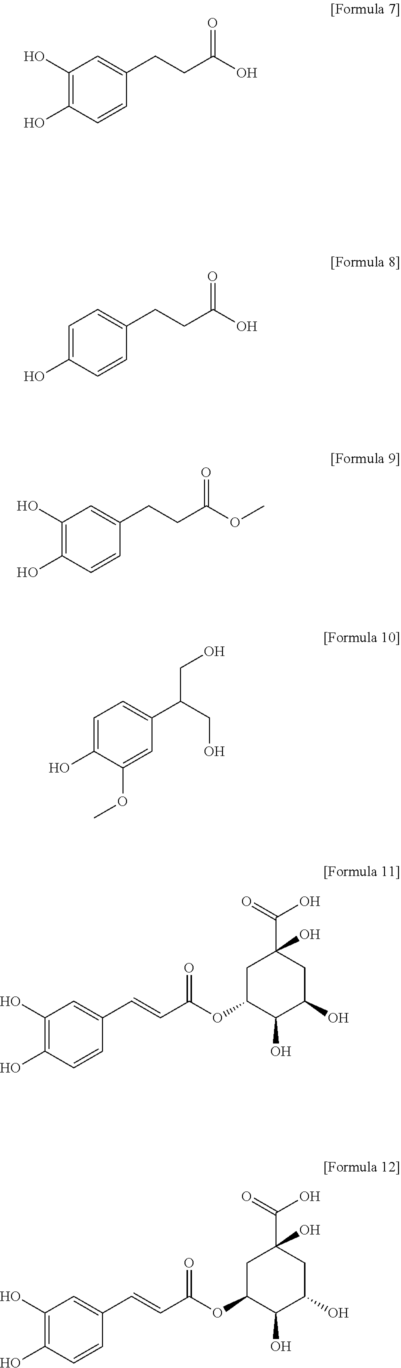 [SMILES: O=C(O)CCc1ccc(O)c(O)c1 ; O=C(O)CCc1ccc(O)cc1 ; COC(=O)CCc1ccc(O)c(O)c1 ; COc1cc(C(CO)CO)ccc1O ; O=C(/C=C/c1ccc(O)c(O)c1)O[C@@H]1C[C@](O)(C(=O)O)C[C@@H](O)[C@H]1O ; O=C(/C=C/c1ccc(O)c(O)c1)O[C@H]1C[C@](O)(C(=O)O)C[C@H](O)[C@H]1O] 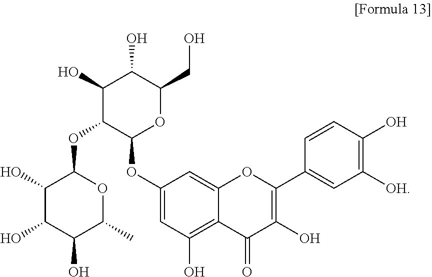 [SMILES: C[C@H]1O[C@H](O[C@H]2[C@H](Oc3cc(O)c4c(=O)c(O)c(-c5ccc(O)c(O)c5)oc4c3)O[C@H](CO)[C@@H](O)[C@@H]2O)[C@@H](O)[C@@H](O)[C@@H]1O]


SEQUENCE LISTING (16 sequences)

SEQ ID NO: 1 - DNA, 20 bp, synthetic construct
  misc_feature 1..20: note = Mstn_primer F
  source 1..20: mol_type = other DNA
tggctcctactggacctctc

SEQ ID NO: 2 - DNA, 20 bp, synthetic construct
  misc_feature 1..20: note = Mstn_primer R
  source 1..20: mol_type = other DNA
aagatgcagcagtcactccc

SEQ ID NO: 3 - DNA, 20 bp, synthetic construct
  misc_feature 1..20: note = Myd1_primer F
  source 1..20: mol_type = other DNA
catagacttgacaggccccg

SEQ ID NO: 4 - DNA, 20 bp, synthetic construct
  misc_feature 1..20: note = Myd1_primer R
  source 1..20: mol_type = other DNA
cgggtccaggtcctcaaaaa

SEQ ID NO: 5 - DNA, 20 bp, synthetic construct
  misc_feature 1..20: note = Myog_primer F
  source 1..20: mol_type = other DNA
agctatccggttccaaagcc

SEQ ID NO: 6 - DNA, 20 bp, synthetic construct
  misc_feature 1..20: note = Myog_primer R
  source 1..20: mol_type = other DNA
gcacaggagaccttggtcag

SEQ ID NO: 7 - DNA, 20 bp, synthetic construct
  misc_feature 1..20: note = Myf5_primer F
  source 1..20: mol_type = other DNA
tccaggtattcccacctgct

SEQ ID NO: 8 - DNA, 20 bp, synthetic construct
  misc_feature 1..20: note = Myf5_primer R
  source 1..20: mol_type = other DNA
tcagctttgtgtgctccgaa

SEQ ID NO: 9 - DNA, 20 bp, synthetic construct
  misc_feature 1..20: note = Ppargc1a_primer F
  source 1..20: mol_type = other DNA
gttgcctgcatgagtgtgtg

SEQ ID NO: 10 - DNA, 20 bp, synthetic construct
  misc_feature 1..20: note = Ppargc1a_primer R
  source 1..20: mol_type = other DNA
cacatgtcccaagccatcca

SEQ ID NO: 11 - DNA, 20 bp, synthetic construct
  misc_feature 1..20: note = Ucp3_primer F
  source 1..20: mol_type = other DNA
gttttgcggacctcctcact

SEQ ID NO: 12 - DNA, 20 bp, synthetic construct
  misc_feature 1..20: note = Ucp3_primer R
  source 1..20: mol_type = other DNA
ctctgtgcgcaccatagtca

SEQ ID NO: 13 - DNA, 20 bp, synthetic construct
  misc_feature 1..20: note = Tomm20_primer F
  source 1..20: mol_type = other DNA
tgtgcggtgtgttgtctgtt

SEQ ID NO: 14 - DNA, 20 bp, synthetic construct
  misc_feature 1..20: note = Tomm20_primer R
  source 1..20: mol_type = other DNA
taagtgcccagagcacagga

SEQ ID NO: 15 - DNA, 20 bp, synthetic construct
  misc_feature 1..20: note = Tfam_primer F
  source 1..20: mol_type = other DNA
gggaatgtggagcgtgctaa

SEQ ID NO: 16 - DNA, 20 bp, synthetic construct
  misc_feature 1..20: note = Tfam_primer R
  source 1..20: mol_type = other DNA
tgatagacgaggggatgcga